(19) United States
(12) Patent Application Publication
(10) Pub. No.: US 2024/0342497 A1
(43) Pub. Date: Oct. 17, 2024

(54) TRANSCRANIAL MAGNETIC STIMULATION USING ROTATING FIELD VECTORS

(71) Applicant: Wave Neuroscience, Inc., Newport Beach, CA (US)

(72) Inventors: James William Phillips, Fountain Valley, CA (US); Robert Isenhart, Newport Beach, CA (US); Alexander Joseph Ring, Irvine, CA (US)

(21) Appl. No.: 18/638,197

(22) Filed: Apr. 17, 2024

Related U.S. Application Data
(60) Provisional application No. 63/496,498, filed on Apr. 17, 2023.

(51) Int. Cl. A61N 2/00; A61N 2/02

(52) U.S. Cl. CPC A61N 2/006; A61N 2/02

(57) ABSTRACT

A method of treating a subject that includes positioning a plurality of magnetic sources in proximity to a head of the subject, each magnetic source being configured to provide a magnetic field, selecting a phase offset between a first magnetic field provided by a first magnetic source of the plurality of magnetic sources and a second magnetic field provided by a second magnetic source of the plurality of magnetic sources, operating the plurality of magnetic sources with the selected phase offset, and applying the magnetic fields provided by the plurality of magnetic sources to the head of the subject to provide a therapeutic treatment within a target area of the subject's brain, wherein the magnetic fields combine to produce a rotating magnetic field vector in proximity to the target area of the subject's brain.

### CROSS-REFERENCE TO RELATED APPLICATIONS

This application claims priority to and the benefit of U.S. Provisional Patent Application No. 63/496,498, titled “TRANSCRANIAL MAGNETIC STIMULATION USING ROTATING FIELD VECTORS” and filed on Apr. 17, 2023, the entire contents of which is hereby incorporated by reference herein.

### FIELD

The disclosure relates to systems and methods providing transcranial magnetic stimulation (TMS) using rotating field vectors.

### BACKGROUND

Mental disorders generate serious problems for the affected people, their families, and society. Currently, psychiatrists and neurophysiologists treat these disorders with a variety of medications, many of which have significant negative side effects. However, treatment of these disorders with magnetic fields can generate positive therapeutic responses.

In some cases, magnetic fields are generated for transcranial magnetic stimulation (TMS). TMS is a non-invasive procedure that typically uses magnetic field pulses or waves to stimulate nerve cells and neuronal circuitry in the brain to improve certain mental disorders such as schizophrenia, obsessive compulsive disorder (OCD), depression, and others.

In some cases, permanent magnets are rotated to generate the magnetic fields used for TMS treatment. By rotating the permanent magnets, rotating magnetic fields are generated that deliver consistent treatment to different neuronal groups across the brain. However, the physical rotation of the permanent magnets can limit or otherwise degrade performance of the treatment.

### SUMMARY

The disclosure relates generally to systems and methods for providing transcranial magnetic stimulation (TMS) using rotating field vectors.

It is to be understood that any combination of features from the methods disclosed herein and/or from the systems and/or devices disclosed herein may be used together, and/or that any features from any or all of these aspects may be combined with any of the features of the embodiments and/or examples disclosed herein to achieve the benefits as described in this disclosure.

At least one aspect of the present disclosure is directed to a method of treating a subject. The method includes positioning a plurality of magnetic sources in proximity to a head of the subject, each magnetic source being configured to provide a magnetic field, selecting a phase offset between a first magnetic field provided by a first magnetic source of the plurality of magnetic sources and a second magnetic field provided by a second magnetic source of the plurality of magnetic sources, operating the plurality of magnetic sources with the selected phase offset, and applying the magnetic fields provided by the plurality of magnetic sources to the head of the subject to provide a therapeutic treatment within a target area of the subject's brain, wherein the magnetic fields combine to produce a rotating magnetic field vector in proximity to the target area of the subject's brain.

In some embodiments, the plurality of magnetic sources are stationary magnetic sources. In some embodiments, each magnetic source of the plurality of magnetic sources is an electromagnetic coil. In some embodiments, applying the magnetic fields provided by the plurality of magnetic sources to the head of the subject includes inducing electric fields in the subject's brain, wherein the electric fields combine to produce a rotating electric field vector within the target area of the subject's brain. In some embodiments, the magnetic field vector rotates in a first direction and the electric field vector rotates in a second direction. In some embodiments, the second direction is opposite from the first direction. In some embodiments, the method includes adjusting an amplitude of each magnetic field provided by the plurality of magnetic sources to control an amplitude of the rotating magnetic field vector. In some embodiments, the amplitude of each magnetic field is adjusted such that the amplitude of the rotating magnetic field vector is substantially constant.

In some embodiments, the method includes selecting a phase offset between the second magnetic field provided by the second magnetic source of the plurality of magnetic sources and a third magnetic field provided by a third magnetic source of the plurality of magnetic sources. In some embodiments, the phase offset between the first magnetic field and the second magnetic field is substantially the same as the phase offset between the second magnetic field and the third magnetic field. In some embodiments, the rotating magnetic field vector rotates in a plane. In some embodiments, positioning a plurality of magnetic sources in proximity to the head of the subject includes arranging the plurality of magnetic sources based on the target area of the subject's brain. In some embodiments, the method includes receiving, via at least one controller, an indication of the target area of the subject's brain. In some embodiments, each magnetic field provided by the plurality of magnetic sources has a sinusoidal waveform. In some embodiments, the therapeutic treatment includes transcranial magnetic stimulation (TMS).

Another aspect of the present disclosure is directed to a system for providing treatment to a subject. The system includes a plurality of magnetic sources configured to be positioned in proximity to a head of the subject, at least one memory storing computer-executable instructions, and at least one processor for executing the instructions stored on the memory. Execution of the instructions causes the at least one processor to select a phase offset between a first magnetic field provided by a first magnetic source of the plurality of magnetic sources and a second magnetic field provided by a second magnetic source of the plurality of magnetic sources and operate the plurality of magnetic sources with the selected phase offset, wherein the magnetic fields provided by the plurality of magnetic sources, when applied to the head of the subject, provide a therapeutic treatment within a target area of the subject's brain by combining to produce a rotating magnetic field vector in proximity to the target area of the subject's brain.

In some embodiments, the plurality of magnetic sources are stationary magnetic sources. In some embodiments, each magnetic source of the plurality of magnetic sources is an electromagnetic coil. In some embodiments, the magnetic fields provided by the plurality of magnetic sources, when applied to the head of the subject, induce electric fields in the subject's brain that combine to produce a rotating electric field vector within the target area of the subject's brain. In some embodiments, the magnetic field vector rotates in a first direction and the electric field vector rotates in a second direction. In some embodiments, the second direction is opposite from the first direction. In some embodiments, execution of the instructions causes the at least one processor to adjust an amplitude of each magnetic field provided by the plurality of magnetic sources to control an amplitude of the rotating magnetic field vector. In some embodiments, the amplitude of each magnetic field is adjusted such that the amplitude of the rotating magnetic field vector is substantially constant.

In some embodiments, execution of the instructions causes the at least one processor to select a phase offset between the second magnetic field provided by the second magnetic source of the plurality of magnetic sources and a third magnetic field provided by a third magnetic source of the plurality of magnetic sources. In some embodiments, the phase offset between the first magnetic field and the second magnetic field is substantially the same as the phase offset between the second magnetic field and the third magnetic field. In some embodiments, the rotating magnetic field vector rotates in a plane. In some embodiments, the plurality of magnetic sources are configured to be positioned in proximity to the head of the subject in an arrangement based on the target area of the subject's brain. In some embodiments, the at least one processor is configured to receive an indication of the target area of the subject's brain. In some embodiments, each magnetic field provided by the plurality of magnetic sources has a sinusoidal waveform. In some embodiments, the therapeutic treatment includes transcranial magnetic stimulation (TMS).

### BRIEF DESCRIPTION OF THE DRAWINGS

The accompanying drawings, which are included to provide a better understanding of the disclosure, illustrate embodiments of the disclosure and together with the description serve to explain the principle of the disclosure. In the drawings:

FIG. 1 illustrates an example diametrically magnetized cylindrical permanent magnet;

FIG. 2 illustrates an example diametrically magnetized cylindrical permanent magnet;

FIG. 3 illustrates an example electromagnetic coil;

FIG. 4A illustrates an example single coil arrangement;

FIG. 4B illustrates an example double coil arrangement;

FIG. 5 illustrates an example plurality of axons;

FIG. 6 illustrates an example of two magnetic field generators interacting;

FIG. 7 illustrates an electromagnetic coil arrangement in accordance with aspects described herein;

FIG. 8 illustrates magnetic field waveforms in accordance with aspects described herein;

FIG. 9A illustrates an electromagnetic coil arrangement in accordance with aspects described herein;

FIG. 9B illustrates a rotating magnetic field vector in accordance with aspects described herein;

FIG. 10 illustrates magnetic field waveforms in accordance with aspects described herein;

FIG. 11 illustrates a method for providing therapeutic treatment using rotating field vectors in accordance with aspects described herein; and

FIG. 12 illustrates an example computer system.

### DETAILED DESCRIPTION

The disclosure relates to systems and methods for providing transcranial magnetic stimulation (TMS) using rotating field vectors.

As described above, magnetic fields are generated for TMS treatments. TMS is a non-invasive procedure that typically uses magnetic field pulses or waves to stimulate nerve cells and neuronal circuitry in the brain to improve certain mental disorders such as schizophrenia, obsessive compulsive disorder (OCD), depression, and others. In some cases, permanent magnets are rotated to generate the magnetic fields used for TMS treatment. By rotating the permanent magnets, rotating magnetic fields are generated that deliver consistent treatment to different neuronal groups across the brain.

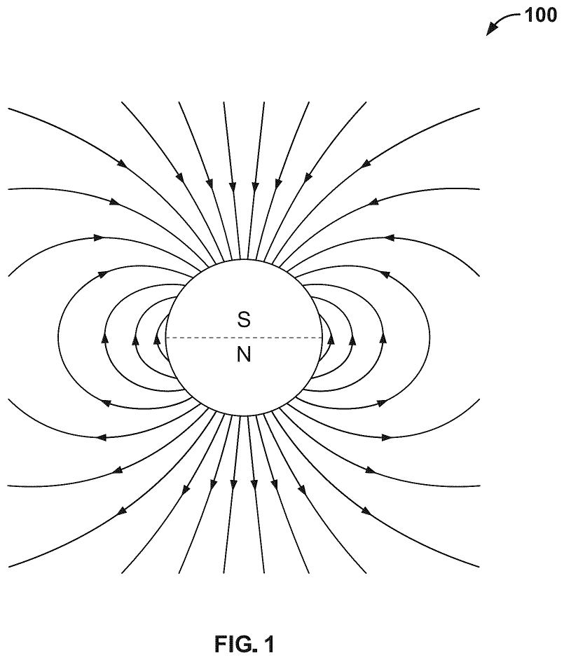

FIG. 1 illustrates an example of a diametrically magnetized cylindrical permanent magnet 100. A magnetic field is generated when the magnet 100 is rotated. When measured at a point near the rounded surface of the magnet 100, the magnetic field is represented by a three-dimensional (3D) vector that rotates in two dimensions along a plane parallel to the magnet's flat surface (e.g., top or bottom surface). In some examples, the lines of the magnetic field travel north to south.

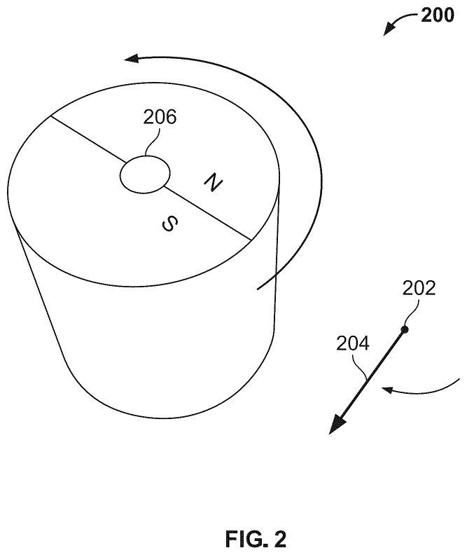

FIG. 2 illustrates an example of a diametrically magnetized cylindrical permanent magnet 200. As shown, the magnet 200 is rotated counter-clockwise. When measured at a point near the rounded surface of the magnet 200, the magnetic field is represented by a field vector that rotates in the opposite direction. For example, the magnetic field vector 202 measured at the point 204 near the curved surface of the magnet 200 rotates clockwise as the magnet 200 turns counter-clockwise. In some examples, the magnet 200 includes a hole 206 where a shaft (not shown) is inserted to rotate the magnet 200. The hole 206 does not significantly affect the magnetic field lines generated by the magnet 200.

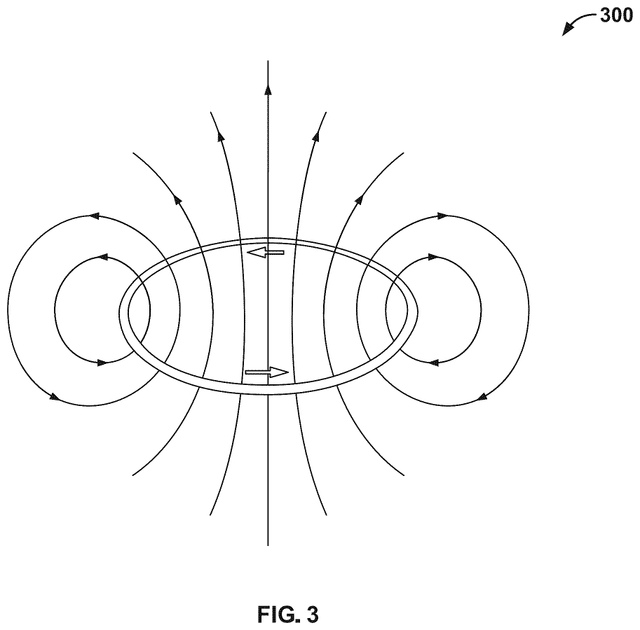

FIG. 3 illustrates an example of an electromagnetic coil 300. In some examples, the magnetic field vector of the coil 300 has a fixed direction. In other words, if the coil 300 is stationary, the magnetic field generated by the coil 300 has a constant orientation. In some examples, the amplitude of the magnetic field is adjusted based on the amplitude of the electric current in the coil 300.

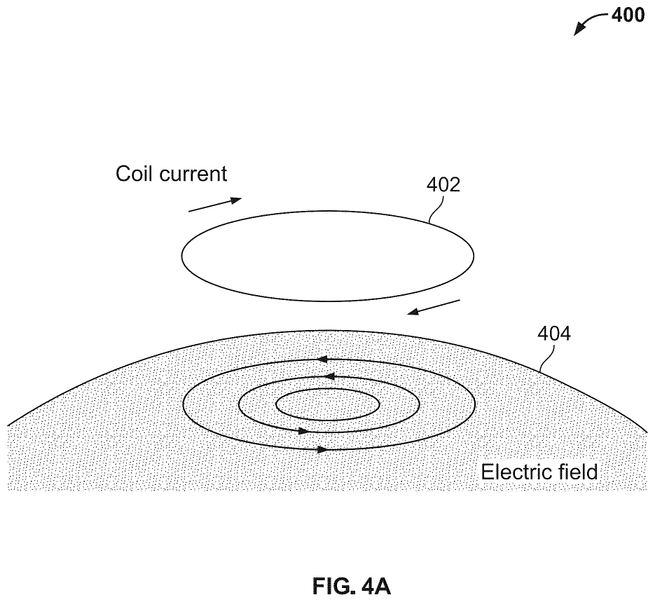

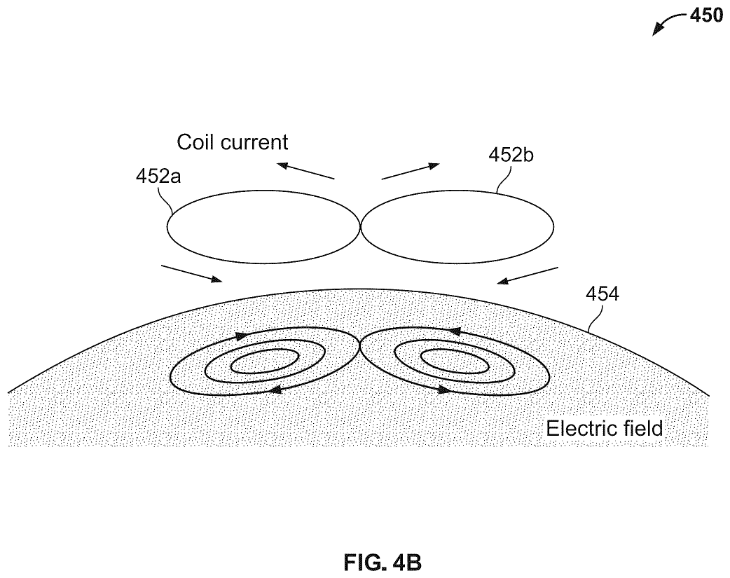

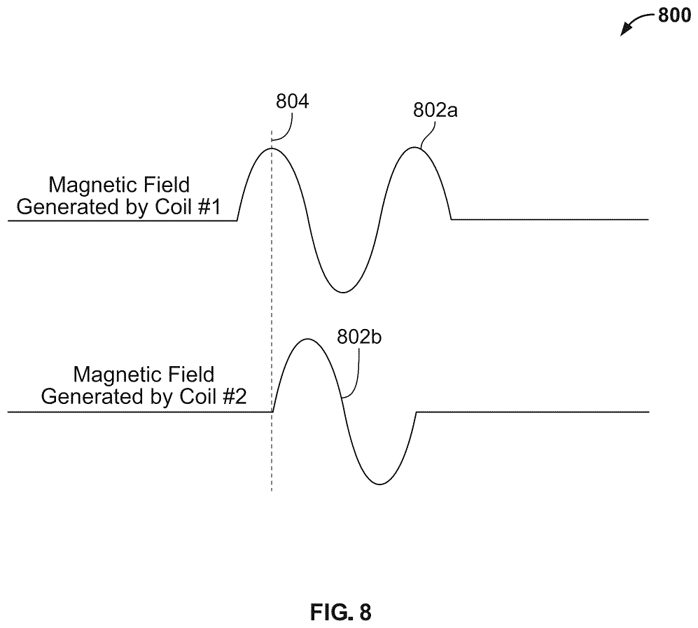

In some examples, electromagnetic coils (e.g., coil 300) are used to produce time-changing magnetic fields that induce electric current flow in the brain. In some examples, the current flow in the brain encircles the magnetic field lines of the time-changing magnetic field(s). FIG. 4A shows an example of a single coil arrangement 400. The arrangement 400 includes a single coil 402 positioned near the head of a subject 404. As shown, the current in the coil 402 travels in a clockwise direction, inducing an electric field in the brain of the subject 404 having a counter-clockwise direction. In some examples, the current in the coil 403 travels in a counter-clockwise direction, inducing an electric field in the brain of the subject 404 having a clockwise direction. Similarly, FIG. 4B illustrates an example of a double (or FIG. 8) coil arrangement 450. The arrangement 450 includes a first coil 452a adjacent to a second coil 452b. The electric current in the first coil 452a travels in a first direction (e.g., counter-clockwise) and the electric current in the second coil 452b travels in a second, opposite direction (e.g., clockwise). In some examples, the arrangement 450 provides induced electric field vectors that add together to create a higher, focused current in the region where the coils 452a, 452b are joined (or closest).

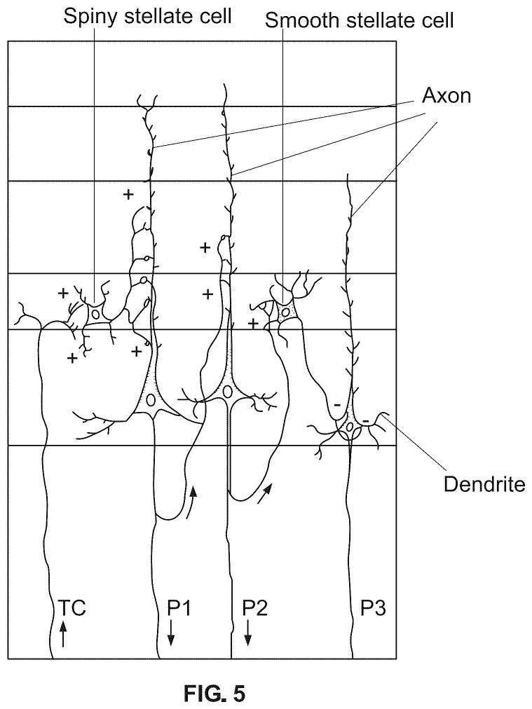

In the examples of FIGS. 4A and 4B, the direction of the induced electric field(s) is stationary. In other words, at a particular point in the brain, the induced electric field has a fixed direction. In some examples, the amplitude and/or the polarity of the induced electric field(s) is adjustable. In some examples, the electric field(s) induce significant current flow in axons and/or dendrites of the brain that run approximately parallel to the direction of the electric field(s). As shown in FIG. 5, axons that are part of pyramidal cells (e.g., cortical neurons) tend to lie parallel to each other and perpendicular to the cortical surface. Due to sulci in the cortex, the axonal direction of cortical neuronal groups tends to vary across the brain. Therefore, if the induced electric field is unidirectional (e.g., stationary) at a certain point in the cortex, the significance (or effectiveness) of a TMS pulse will vary across neurons. In some examples, to ensure that each neuron is affected by the electric field(s) induced from the time-changing magnetic field(s), the magnetic field vector direction is changed over time (e.g., not just the amplitude of the magnetic field vector).

The magnetic field of a rotating magnet (e.g., magnet 100 of FIG. 1 or magnet 200 of FIG. 2) has a magnetic field vector direction that changes over time. When looking at a single point in the brain beneath the rotating magnet, the direction of the field change rotates about the axis perpendicular to the curved surface of the rotating magnet. Therefore, the induced electric field at that point changes direction over time. This change in direction allows the induced electric field to affect far more neuronal axons and dendrites, because the electric field is not always perpendicular to the direction of each axon and/or dendrite.

In some examples, the potential effect on the brain of the rotating diametrically magnetized permanent magnet is greater (or at least different) than the effect of sending a pulse of current through a stationary wire (e.g., the electromagnetic coil 300 of FIG. 3). However, the rotating magnet is limited to a maximum speed of rotation. Likewise, in some examples, it is difficult to shape the magnetic field waveform of the rotating magnet. For example, the maximum rotation speed for a Neodymium cylindrical magnet is based on the maximum motor speed as well as the point at which the centrifugal forces will cause the magnet to break apart. In some examples, a high-end motor can rotate at up to 30,000 rpm, which equates to 500 revolutions per second, generating a 500 Hz waveform with a 2 msec period. This period is significantly greater than the pulse width of approximately 300 usec generated by a TMS coil (e.g., coil 300). In some examples, the rotating magnet cannot easily generate a single pulse, effectively turning the magnet through a single full rotation and then stopping. In some examples, momentum from the magnet rotation makes a single cycle at only a few milliseconds difficult to achieve. In addition, the inclusion of motors and other components (e.g., shafts) can increase the size and weight of treatment devices.

Accordingly, systems and methods for providing TMS using rotating field vectors are provided herein. In some examples, improved TMS performance is achieved through the use of stationary magnets (e.g., electromagnetic coils) that produce rotating magnetic field vectors. In at least one embodiment, a plurality of magnetic sources are positioned in proximity to a head of the subject and each magnetic source is configured to provide a magnetic field. In some examples, a phase offset is selected between a first magnetic field provided by a first magnetic source of the plurality of magnetic sources and a second magnetic field provided by a second magnetic source of the plurality of magnetic sources. In some examples, the plurality of magnetic sources are operated with the selected phase offset. The magnetic fields provided by the plurality of magnetic sources are applied to the head of the subject to provide a therapeutic treatment within a target area of the subject's brain. In some examples, the magnetic fields combine to produce a rotating magnetic field vector in proximity to the target area of the subject's brain.

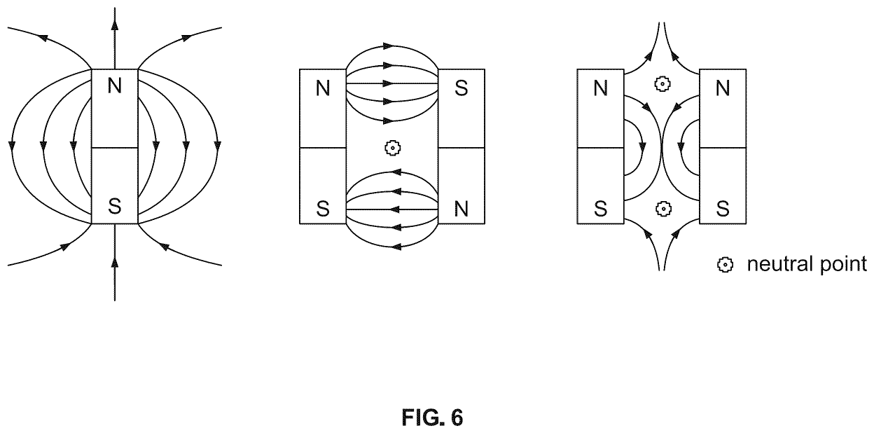

Turning to FIG. 6, when two magnetic field generators (e.g., magnets) are brought into proximity, the resulting magnetic field vector at any point is equal to the addition of the magnetic field vectors from the two magnetic field sources. For example, the arrows in FIG. 6 show the modified magnetic field produced by two nearby magnetic field generators. The neutral points are created by the vectors from each magnetic field vector opposing each other and summing to zero.

As such, if two electromagnetic coils are placed in proximity to each other, and each generates a magnetic field pulse at the same instant, a non-rotating magnetic field pulse is generated. In other words, when measured at a point between the two coils, the magnetic field is represented by a non-rotating magnetic field vector. In such examples, the amplitude and direction of the non-rotating magnetic field vector are determined by the sum of the constituent magnetic field vectors from each coil. However, if the magnetic pulse is generated with a phase offset between the two coils, the result at the same point is a rotating magnetic field vector. In some examples, the magnetic field vector rotates in a plane formed by the vectors of the magnetic fields generated by the coils.

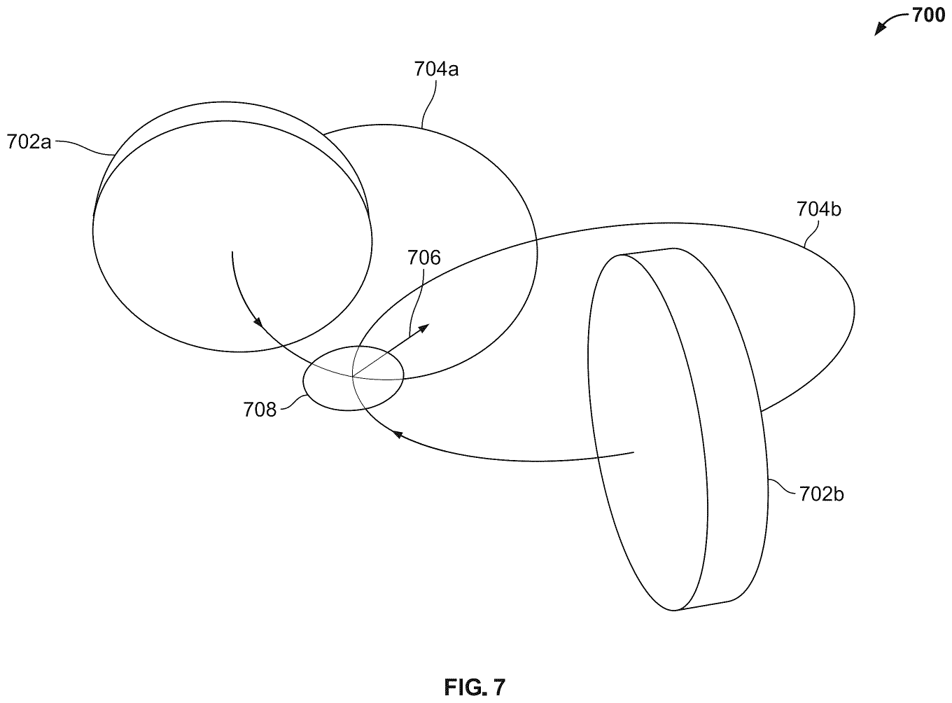

FIG. 7 illustrates an electromagnetic coil arrangement 700 in accordance with aspects described herein. In some examples, the arrangement 700 includes a first coil 702a and a second coil 702b. In some examples, the arrangement 700 is configured to be included in or coupled to a treatment device. In some examples, the first coil 702a is configured to receive a first current signal to produce a first magnetic field and the second coil 702b is configured to receive a second current signal to produce a second magnetic field. In some examples, the coils 702a, 702b are configured to be in communication with at least one controller or processor (e.g., a controller of the treatment device). In some examples, the controller or processor is configured to provide the first and second current signals to the first and second coils 702a, 702b. In some examples, the controller or processor is configured to operate additional circuitry to provide the first and second current signals.

As shown, a first oval magnetic field vector 704a corresponds to the magnetic field provided by the first coil 702a. Likewise, a second oval magnetic field vector 704b corresponds to the magnetic field provided by the second coil 702b. At the intersection is a magnetic field vector 706 corresponding to the sum of the two constituent vectors 704a, 704b. The circle 708 represents the plane of rotation for the vector 706. In some examples, the direction of the vector 706 is dependent on the amplitude and polarity of the two constituent vectors 704a, 704b at the location of the vector 706 (e.g., the intersection point within the circle 708).

In some examples, both coils 702a, 702b generate a magnetic field that consists of one or more cycles of a sinusoid. In some examples, the sinusoids have a 90-degree phase delay (or offset) relative to each other. In such examples, the vector 706 will be in the form of an ellipse while rotating, where the vertices of the ellipse are defined by the amplitude of the magnetic fields generated by each coil 702a, 702b and the position of the measurement point relative to the two coils 702a, 702b. In some examples, the magnetic field amplitudes are adjusted such that the vector 706 moves in a circle with a constant amplitude (e.g., by adjusting the amplitudes of the coil currents).

FIG. 8 illustrates a plot 800 of example magnetic field waveforms in accordance with aspects described herein. The plot 800 includes a first waveform 802a corresponding to the magnetic field generated by the first coil 702a and a second waveform 802b corresponding to the magnetic field generated by the second coil 702b. As shown, the second waveform 802b is offset by 90 degrees from the first waveform 802a. For example, at the reference marker 804, the first waveform 802a is at a peak while the second waveform 802b is at a zero-crossing, indicating a phase offset (or shift) of 90 degrees. It should be appreciated that different phase offsets (or shifts) may be used (e.g., 45 degrees, 60 degrees, etc.) between the waveforms 802a, 802b. In some examples, the phase offset amount between the waveforms 802a, 802b determines the shape of the rotating vector 706 (e.g., a circle, an ellipse, etc.). In addition, in some examples, the first waveform 802a is offset from the second waveform 802b (e.g., the second waveform 802b leads the first waveform 802a).

As described above, the magnetic field vector 706 rotates within a plane of rotation (e.g., the circle 708). In some examples, the plane of rotation corresponds to a two-dimensional (2D) plane, such as the x-y plane, the x-z plane, or the y-z plane. In some examples, the plane of rotation is determined by the orientation and position of the coils 702a, 702b. In some examples, the plane of rotation can be assigned to any 3D plane (e.g., rotation in the x, y, and/or z directions) by using three or more electromagnetic coils.

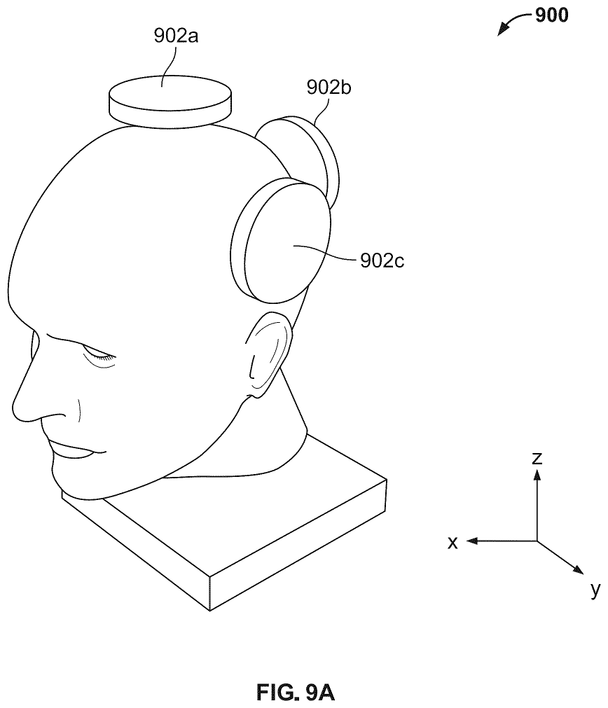

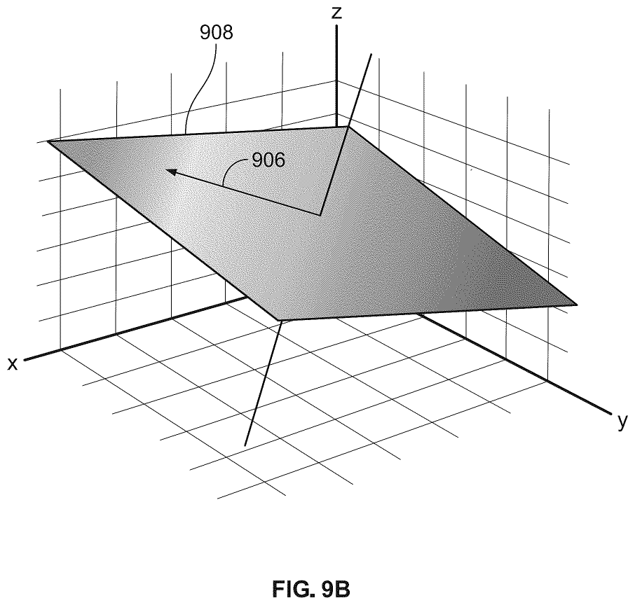

FIG. 9A illustrates an electromagnetic coil arrangement 900 in accordance with aspects described herein. In some examples, the arrangement 900 includes a first coil 902a, a second coil 902b, and a third coil 902c. In some examples, the arrangement 900 is configured to be included in or coupled to a treatment device. In some examples, the first coil 902a is configured to receive a first current signal to produce a first magnetic field, the second coil 902b is configured to receive a second current signal to produce a second magnetic field, and the third coil 902c is configured to receive a third current signal to produce a third magnetic field. In some examples, the coils 902a, 902b, 902c are configured to be in communication with at least one controller or processor (e.g., a controller of the treatment device). In some examples, the controller or processor is configured to provide the first, second, and third current signals to the coils 902a, 902b, 902c. In some examples, the controller or processor is configured to operate additional circuitry to provide the first, second, and third current signals.

In some examples, coils 902a, 902b, 902c each generate a magnetic field that consists of one or more cycles of a sinusoid. In some examples, the sinusoids have a 60-degree phase delay (or offset) relative to each other. For example, the second coil 902b may be configured to generate a magnetic field that is offset by 60 degrees from the magnetic field generated by the first coil 902a. Likewise, the third coil 902c may be configured to generate a magnetic field that is offset by 60 degrees from the magnetic field generated by the second coil 902b (or 120 degrees offset from the magnetic field generated by the first coil 902a). Like the arrangement 700 of FIG. 7, the magnetic fields generated by the coils 902a, 902b, 902c combine to provide a rotating magnetic field vector.

In some examples, the rotating magnetic field vector provided by the three coils 902a, 902b, 902c rotates in a 3D plane. FIG. 9B illustrates an example rotating magnetic field vector 906. In some examples, the rotating vector 906 corresponds to a vector of the combined magnetic field provided by the electromagnetic coil arrangement 900. As shown, the vector 906 rotates within a three-dimensional plane 908. In some examples, the vector 906 will be in the form of an ellipse while rotating, where the vertices of the ellipse are defined by the amplitude of the magnetic fields generated by each coil 902a, 902b, 906c and the position of the measurement point relative to the three coils 902a, 902b, 906c. In some examples, the magnetic field amplitudes are adjusted such that the vector 906 moves in a circle with a substantially constant amplitude. In some examples, to rotate the vector 906 in a 2D plane (e.g., the x-y plane), one of the coils 902a, 902b, or 902c is disabled (e.g., does not generate a magnetic field).

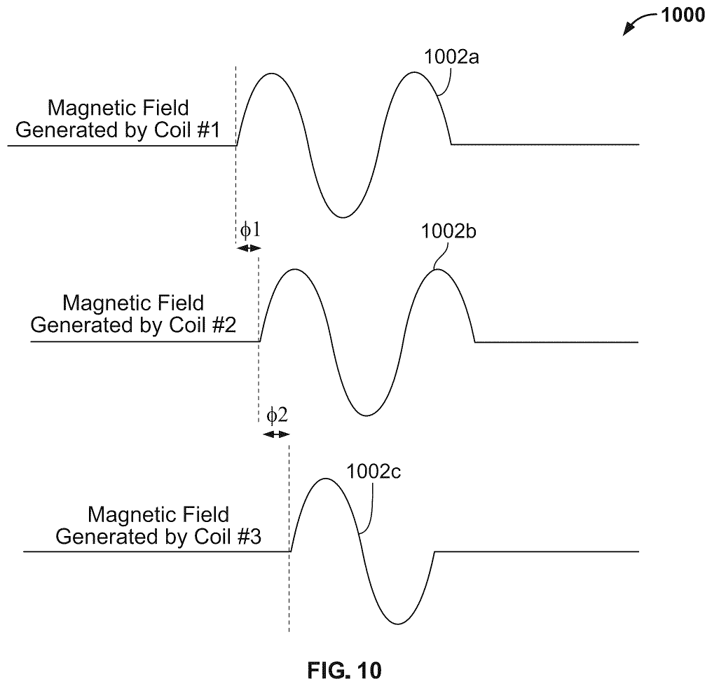

FIG. 10 illustrates a plot 1000 of example magnetic field waveforms in accordance with aspects described herein. The plot 1000 includes a first waveform 1002a corresponding to the magnetic field generated by the first coil 902a, a second waveform 1002b corresponding to the magnetic field generated by the second coil 902b, and a third waveform 1002c corresponding to the magnetic field generated by the third coil 902c. As shown, the second waveform 1002b is offset by $1 degrees from the first waveform 1002a. Likewise, the third waveform 1002c is offset by $2 degrees from the second waveform 1002b. In some examples, $1 and $2 both have values of 60 degrees. It should be appreciated that different phase offsets (or shifts) may be used (e.g., 45 degrees, 90 degrees, etc.) between the waveforms 1002a, 1002b, 1002c. In some examples, the phase offset amount between the waveforms 1002a, 1002b, 1002c determines the shape of the rotating vector 906 (e.g., a circle, an ellipse, etc.). In addition, in some examples, the first waveform 1002a is offset from the second waveform 1002b (e.g., the second waveform 1002b leads the first waveform 1002a) and/or the second waveform 1002b is offset from the third waveform 1002c (e.g., the third waveform 1002c leads the second waveform 1002b).

In some examples, the magnetic field vector (e.g., vector 706 or vector 906) is moved to a point in the brain where stimulation is desired. In some examples, using MRI, it is determined whether the stimulation point is in a sulcus of the cortex or if it is near the outer surface of the brain, giving an indication of the direction of the axons in that region. The electromagnetic coils (e.g., of coil arrangement 700 or 900) are operated such that the rotating magnetic field vector forms an ellipse that delivers maximum power to the stimulation point. In some examples, the amplitudes of the magnetic fields generated by each coil are adjusted such that the resulting vector is stationary in any desired direction, with only the amplitude varying. In some examples, by rotating the magnetic field vector in the desired ellipse, an electric current is induced precisely in-line with a specified set of axons at the stimulation point. In some examples, the amplitude and polarity of each magnetic field (e.g., each magnetic field pulse) is adjusted such that the axis of rotation for the magnetic field at the desires stimulation point is parallel to the direction of the axons for the neurons being stimulated.

As described above, in some examples, the desired stimulation point is determined using an MRI. In some examples, the desired stimulation point is determined using an EEG waveform. In some examples, the desired stimulation point is determined using at least one of an electrocardiogram (ECG) recording, an MRI image, a Brain Score, an EKG recording, a SPECT scan, a PET scan, x-ray, CT scan, Ultrasound, mammogram, Flouroscopy, arthrogram, myelogram, DEXA bone density scan, body temperature, respiratory rate, heart rate, blood pressure, blood oxygen saturation, Complete Blood Count, basic metabolic panel, comprehensive metabolic panel, lipid panel, liver panel, thyroid stimulating hormone, hemoglobin A1CProthrombin time, blood enzyme tests, blood clotting test, urinalysis, cultures, applanation tonometry, corneal topography, Fluorescein angiogram, slit-lamp exam image, retinal tomography, visual acuity testing, visual field test results, mental health assessment, behavioral health assessment, psychiatric assessment, athletic performance measurement, academic performance measurement, intelligence test result, self-assessment, demographics, and personality profile.

In some examples, the plane of rotation of the rotating magnetic field vector (e.g., vector 706 or vector 906) is varied to affect a greater portion of neurons in the brain. In some examples, each magnetic stimulation pulse is adjusted such that the plane of rotation is orthogonal to the previous two planes of rotation (e.g., of the previous two magnetic stimulation pulses), allowing all neurons to be affected significantly by at least one magnetic stimulation pulse.

As described above, the use of sinusoidal magnetic pulses enables the magnetic field rotation to be elliptical. However, in some examples, different geometries for the magnetic field rotation are applied by changing the waveform of the magnetic field (e.g., square wave, sawtooth wave, frequency chirp waveforms, etc.). In some examples, the waveform of the magnetic field corresponds to the waveform of the current provided to the electromagnetic coil.

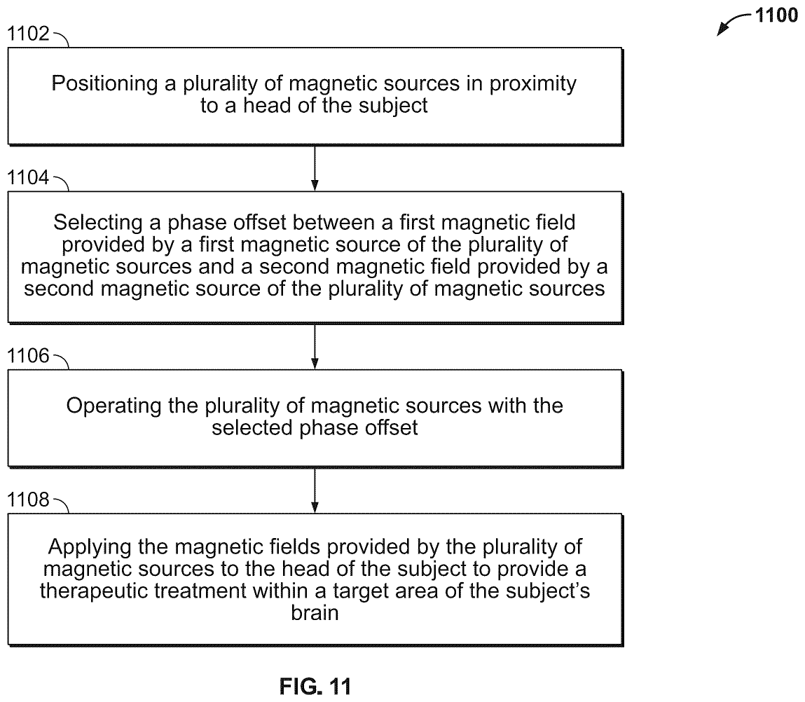

FIG. 11 is a flow diagram of a method 1100 for providing therapeutic treatment using rotating field vectors in accordance with aspects described herein. In some examples, the method 1100 is configured to be carried out using the electromagnetic coil arrangement 700 of FIG. 7 or the electromagnetic coil arrangement 700 of FIG. 9.

At block 1102, a plurality of magnetic sources are positioned in proximity to the head of the subject. As described above, each magnetic source is configured to provide a magnetic field. In some examples, the plurality of magnetic sources are stationary magnetic sources. In some examples, each magnetic source of the plurality of magnetic sources is an electromagnetic coil (e.g., coils 702a, 702b or coils 902a, 902b, 902c). In some examples, the plurality of magnetic sources are arranged based on a target area (e.g., a desired stimulation point) of the subject's brain. In some examples, an indication of the target area is received via at least one controller or processor in communication with the plurality of magnetic sources. In some examples, a user selects the target area via a user interface.

At block 1104, a phase offset is selected between a first magnetic field provided by a first magnetic source of the plurality of magnetic sources and a second magnetic field provided by a second magnetic source of the plurality of magnetic sources. In some examples, a phase offset is selected between the second magnetic field provided by the second magnetic source of the plurality of magnetic sources and a third magnetic field provided by a third magnetic source of the plurality of magnetic sources. In some examples, the phase offset between the first magnetic field and the second magnetic field is substantially the same as the phase offset between the second magnetic field and the third magnetic field.

At block 1106, the plurality of magnetic sources are operated with the selected phase offset(s). In some examples, the plurality of magnetic sources are operated to provide magnetic fields having sinusoidal waveforms.

At block 1108, the magnetic fields provided by the plurality of magnetic sources are applied to the head of the subject to provide a therapeutic treatment within the target area of the subject's brain. The magnetic fields combine to produce a rotating magnetic field vector in proximity to the target area of the subject's brain. In some examples, the magnetic fields provided by the plurality of magnetic sources induce electric fields in the subject's brain. The electric fields combine to produce a rotating electric field vector within the target area of the subject's brain. In some examples, the magnetic field vector rotates in a first direction and the electric field vector rotates in a second direction. In some examples, the magnetic field vector and the electric field vector rotate in opposite directions.

In some examples, the amplitude of each magnetic field provided by the plurality of magnetic sources is adjusted to control an amplitude of the rotating magnetic field vector. In some examples, the amplitudes are adjusted by controlling an amplitude of the current provided to each coil. In some examples, the amplitude of each magnetic field is adjusted such that the amplitude of the rotating magnetic field vector (or the rotating electric field vector) is substantially constant. In some examples, the rotating magnetic field vector (or the rotating electric field vector) rotates in a plane (e.g., a 2D plane or a 3D plane).

As described above, the electromagnetic coil arrangements 700 and 900 are configured to be included in or coupled to a treatment device that provides treatment (e.g., brain stimulation) to the subject's brain. In some examples, the treatment device is configured to provide magnetic brain stimulation (e.g., TMS or repetitive TMS). In some examples, the treatment corresponds to a treatment plan (or treatment settings). In some examples, the treatment is directed to improving the symptoms of Autism Spectrum Disorder, Alzheimer's disease, ADHD, schizophrenia, anxiety, depression, coma, Parkinson's disease, substance abuse, bipolar disorder, sleep disorder, eating disorder, tinnitus, traumatic brain injury, post-traumatic stress disorder, or fibromyalgia. In some examples, the treatment device is configured to be worn on the subject's head while receiving treatment. In some examples, the treatment device provides sensory stimulation including flashing light, sound, video, or touch.

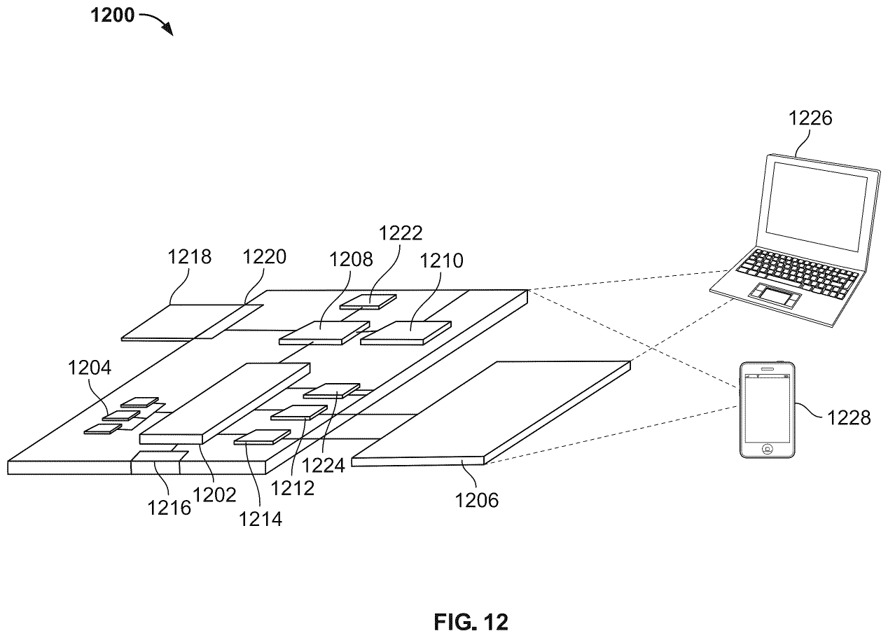

FIG. 12 shows an example of a generic computing device 1200, which may be used with some of the techniques described in this disclosure (e.g., to control or operate the coil arrangements 700, 900 of FIGS. 7 and 9). Computing device 1200 includes a processor 1202, memory 1204, an input/output device such as a display 1206, a communication interface 1208, and a transceiver 1210, among other components. The device 1200 may also be provided with a storage device, such as a micro-drive or other device, to provide additional storage. Each of the components 1200, 1202, 1204, 1206, 1208, and 1210, are interconnected using various buses, and several of the components may be mounted on a common motherboard or in other manners as appropriate.

The processor 1202 can execute instructions within the computing device 1200, including instructions stored in the memory 1204. The processor 1202 may be implemented as a chipset of chips that include separate and multiple analog and digital processors. The processor 1202 may provide, for example, for coordination of the other components of the device 1200, such as control of user interfaces, applications run by device 1200, and wireless communication by device 1200.

Processor 1202 may communicate with a user through control interface 1212 and display interface 1214 coupled to a display 1206. The display 1206 may be, for example, a TFT LCD (Thin-Film-Transistor Liquid Crystal Display) or an OLED (Organic Light Emitting Diode) display, or other appropriate display technology. The display interface 1214 may comprise appropriate circuitry for driving the display 1206 to present graphical and other information to a user. The control interface 1212 may receive commands from a user and convert them for submission to the processor 1202. In addition, an external interface 1216 may be provided in communication with processor 1202, so as to enable near area communication of device 1200 with other devices. External interface 1216 may provide, for example, for wired communication in some implementations, or for wireless communication in other implementations, and multiple interfaces may also be used.

The memory 1204 stores information within the computing device 1200. The memory 1204 can be implemented as one or more of a computer-readable medium or media, a volatile memory unit or units, or a non-volatile memory unit or units. Expansion memory 1218 may also be provided and connected to device 1200 through expansion interface 1220, which may include, for example, a SIMM (Single In Line Memory Module) card interface. Such expansion memory 1218 may provide extra storage space for device 1200, or may also store applications or other information for device 1200. Specifically, expansion memory 1218 may include instructions to carry out or supplement the processes described above, and may include secure information also. Thus, for example, expansion memory 1218 may be provided as a security module for device 1200, and may be programmed with instructions that permit secure use of device 1200. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1204, expansion memory 1218, memory on processor 1202, or a propagated signal that may be received, for example, over transceiver 1210 or external interface 1216.

Device 1200 may communicate wirelessly through communication interface 1208, which may include digital signal processing circuitry where necessary. Communication interface 1208 may in some cases be a cellular modem. Communication interface 1208 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1210. In addition, short-range communication may occur, such as using a Bluetooth, WiFi, or other such transceiver (not shown). In addition, GPS (Global Positioning System) receiver module 1222 may provide additional navigation- and location-related wireless data to device 1200, which may be used as appropriate by applications running on device 1200.

Device 1200 may also communicate audibly using audio codec 1224, which may receive spoken information from a user and convert it to usable digital information. Audio codec 1224 may likewise generate audible sound for a user, such as through a speaker, e.g., in a handset of device 1200. Such sound may include sound from voice telephone calls, may include recorded sound (e.g., voice messages, music files, etc.) and may also include sound generated by applications operating on device 1200. In some examples, the device 1200 includes a microphone to collect audio (e.g., speech) from a user. Likewise, the device 1200 may include an input to receive a connection from an external microphone.

The computing device 1200 may be implemented in a number of different forms, as shown in FIG. 12. For example, it may be implemented as a computer (e.g., laptop) 1226. It may also be implemented as part of a smartphone 1228, smart watch, tablet, personal digital assistant, or other similar mobile device.

Some implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of treating a subject, comprising:
positioning a plurality of magnetic sources in proximity to a head of the subject, each magnetic source being configured to provide a magnetic field;
selecting a phase offset between a first magnetic field provided by a first magnetic source of the plurality of magnetic sources and a second magnetic field provided by a second magnetic source of the plurality of magnetic sources;
operating the plurality of magnetic sources with the selected phase offset; and
applying the magnetic fields provided by the plurality of magnetic sources to the head of the subject to provide a therapeutic treatment within a target area of the subject's brain, wherein the magnetic fields combine to produce a rotating magnetic field vector in proximity to the target area of the subject's brain.

2. The method of claim 1, wherein the plurality of magnetic sources are stationary magnetic sources.

3. The method of claim 1, wherein each magnetic source of the plurality of magnetic sources is an electromagnetic coil.

4. The method of claim 1, wherein applying the magnetic fields provided by the plurality of magnetic sources to the head of the subject includes inducing electric fields in the subject's brain, wherein the electric fields combine to produce a rotating electric field vector within the target area of the subject's brain.

5. The method of claim 4, wherein the magnetic field vector rotates in a first direction and the electric field vector rotates in a second direction.

6. The method of claim 5, wherein the second direction is opposite from the first direction.

7. The method of claim 1, further comprising:
adjusting an amplitude of each magnetic field provided by the plurality of magnetic sources to control an amplitude of the rotating magnetic field vector.

8. The method of claim 7, wherein the amplitude of each magnetic field is adjusted such that the amplitude of the rotating magnetic field vector is substantially constant.

9. The method of claim 1, further comprising:
selecting a phase offset between the second magnetic field provided by the second magnetic source of the plurality of magnetic sources and a third magnetic field provided by a third magnetic source of the plurality of magnetic sources.

10. The method of claim 9, wherein the phase offset between the first magnetic field and the second magnetic field is substantially the same as the phase offset between the second magnetic field and the third magnetic field.

11. The method of claim 1, wherein the rotating magnetic field vector rotates in a plane.

12. The method of claim 1, wherein positioning a plurality of magnetic sources in proximity to the head of the subject includes arranging the plurality of magnetic sources based on the target area of the subject's brain.

13. The method of claim 1, further comprising:
receiving, via at least one controller, an indication of the target area of the subject's brain.

14. The method of claim 1, wherein each magnetic field provided by the plurality of magnetic sources has a sinusoidal waveform.

15. The method of claim 1, wherein the therapeutic treatment includes transcranial magnetic stimulation (TMS).

16. A system for providing treatment to a subject, comprising:
a plurality of magnetic sources configured to be positioned in proximity to a head of the subject;
at least one memory storing computer-executable instructions; and
at least one processor for executing the instructions stored on the memory, wherein execution of the instructions causes the at least one processor to:
select a phase offset between a first magnetic field provided by a first magnetic source of the plurality of magnetic sources and a second magnetic field provided by a second magnetic source of the plurality of magnetic sources; and
operate the plurality of magnetic sources with the selected phase offset,
wherein the magnetic fields provided by the plurality of magnetic sources, when applied to the head of the subject, provide a therapeutic treatment within a target area of the subject's brain by combining to produce a rotating magnetic field vector in proximity to the target area of the subject's brain.

17. The system of claim 16, wherein the plurality of magnetic sources are stationary magnetic sources.

18. The system of claim 16, wherein each magnetic source of the plurality of magnetic sources is an electromagnetic coil.

19. The system of claim 16, wherein the magnetic fields provided by the plurality of magnetic sources, when applied to the head of the subject, induce electric fields in the subject's brain that combine to produce a rotating electric field vector within the target area of the subject's brain.

20. The system of claim 19, wherein the magnetic field vector rotates in a first direction and the electric field vector rotates in a second direction.

21. The system of claim 20, wherein the second direction is opposite from the first direction.

22. The system of claim 16, wherein execution of the instructions causes the at least one processor to:
adjust an amplitude of each magnetic field provided by the plurality of magnetic sources to control an amplitude of the rotating magnetic field vector.

23. The system of claim 22, wherein the amplitude of each magnetic field is adjusted such that the amplitude of the rotating magnetic field vector is substantially constant.

24. The system of claim 16, wherein execution of the instructions causes the at least one processor to:
select a phase offset between the second magnetic field provided by the second magnetic source of the plurality of magnetic sources and a third magnetic field provided by a third magnetic source of the plurality of magnetic sources.

25. The system of claim 24, wherein the phase offset between the first magnetic field and the second magnetic field is substantially the same as the phase offset between the second magnetic field and the third magnetic field.

26. The system of claim 16, wherein the rotating magnetic field vector rotates in a plane.

27. The system of claim 16, wherein the plurality of magnetic sources are configured to be positioned in proximity to the head of the subject in an arrangement based on the target area of the subject's brain.

28. The system of claim 16, wherein the at least one processor is configured to
receive an indication of the target area of the subject's brain.

29. The system of claim 16, wherein each magnetic field provided by the plurality of magnetic sources has a sinusoidal waveform.

30. The system of claim 16, wherein the therapeutic treatment includes transcranial magnetic stimulation (TMS).
